(19) United States
(12) Patent Application Publication
(10) Pub. No.: US 2011/0217553 A1
(43) Pub. Date: Sep. 8, 2011

(54) Frozen Ionic Liquid Microparticles and Nanoparticles, and Methods for their Synthesis and Use

(76) Inventors: Isiah M. Warner, Baton Rouge, LA (US); Aaron Tesfai, Baton Rouge, LA (US); Bilal M. El-Zahab, Baton Rouge, AL (US); David Bwambok, Baton Rouge, LA (US); Gary A. Baker, Knoxville, TN (US); Sayo O. Fakayode, Baton Rouge, LA (US); Mark Lowa, Fontanelle, IA (US); Michael P. Tolocka, Chapel Hill, NC (US); Sergio De Rooy, Baton Rouge, LA (US)

(21) Appl. No.: 12/808,782

(22) PCT Filed: Dec. 9, 2008
(86) PCT No.: PCT/US08/86065

§ 371 Date: Jun. 17, 2010

Related U.S. Application Data
(60) Provisional application No. 61/015,378, filed on Dec. 20, 2007.
(60) Provisional application No. 61/087,831, filed on Aug. 11, 2008.

(51) Int. Cl. B32B 9/00; B29B 9/06; C08K 5/3445; B82Y 5/00; B82Y 30/00; B82Y 15/00

(52) U.S. Cl. 428/402.24; 264/11; 428/402; 524/106; 977/700; 977/906; 977/773

(57) ABSTRACT

“Frozen ionic liquid” microparticles and nanoparticles are disclosed, as are alternative methods of making the particles. The particles may be monodisperse or polydisperse, with spherical or other shapes. The particles may be prepared without specialized equipment, and without harsh conditions. The microparticles and nanoparticles have uses in biomedical, materials, analytical, and other fields.

(In countries other than the United States:) The benefit of the Dec. 20, 2007 filing date of U.S. provisional patent application 61/015,378 and of the Aug. 11, 2008 filing date of U.S. provisional patent application 61/087,831 is claimed under applicable treaties and conventions. (In the United States:) The benefit of the Dec. 20, 2007 filing date of U.S. provisional patent application 61/015,378 and of the Aug. 11, 2008 filing date of U.S. provisional patent application 61/087,831 is claimed under 35 U.S.C. §119(e).

The development of this invention was partially funded by the United States Government under grant number 1R01GM079670-01A2 awarded by the National Institutes of Health; and under grant number CHE-0616824 awarded by the National Science Foundation. The Government has certain rights in this invention.

### TECHNICAL FIELD

This invention pertains to microparticles and nanoparticles and methods for their synthesis and use, particularly to microparticles and nanoparticles comprising “frozen ionic liquids.”

### BACKGROUND ART

Natural polymers such as proteins and polysaccharides have often been used for drug delivery. However, these polymers can contain impurities. In addition, crosslinking can degrade drug molecules. Synthetic polymers such as poly(lactic acid), poly(lactide-co-glycolide), and polystyrene have also been used for drug delivery and other purposes. Although some of these polymers are biodegradable, they are typically prepared in organic solvent, which has limited their use due to concerns over possible traces of toxic organic solvents, surfactants, and residual monomers. Also, the organic solvents themselves can be a cause for environmental concerns.

Liposomes have also been used for drug delivery. However, liposomes suffer from poor entrapment efficiency and limited stability.

Porous, hollow silica nanoparticles have sometimes been used, because of their thermal stability and compatibility with many other types of materials. The pore structures of these particles produce certain disadvantages. Because the pores are interconnected, the encapsulated molecules can be released randomly. A capping agent is therefore often required, to inhibit untimely release. In addition, silica is not suitable for many applications since it is not biodegradable.

Silica nanoparticles with different porosities and pore sizes have also been used as packing materials in liquid chromatography. However, modifying silica to impart other properties (e.g., hydrophobicity, chirality, etc.) requires lengthy and tedious functionalization procedures, and often the surface is not fully functionalized, especially inside the pores due to diffusional and wetting limitations.

Due to their high luminescence, quantum dots are popular in various systems, including biodetection systems. However, quantum dots are very toxic.

Ionic liquids (ILs) are salts with relatively low melting points. Ionic liquids typically comprise relatively bulky organic cations and diffuse-charge inorganic anions such as PF₆⁻, BF₄⁻, Tf₂N⁻, or NO₃⁻, although in some ILs the anion is organic, or both cation and anion may be organic. Typically, the ions are sterically mismatched, hindering crystal formation. The properties of ILs are highly “tunable,” allowing ready modifications to meet specific needs by simple changes in the cation, the anion, or both. In addition, many ILs have useful properties such as high thermal stability, non-flammability, and essentially zero vapor pressure. With these unique characteristics, many ILs have been regarded as “green” solvents, since their use need not entail emissions of volatile organic compounds (VOCs), as do more traditional industrial solvents.

The feasibility of incorporating chiral centers within IL building blocks has recently sparked interest in the use of ILs as chiral solvents and selectors. For example, chiral ILs have been used as chiral selectors to discriminate between enantiomeric forms of drug molecules. Chiral ILs have also been used as the stationary phase in gas chromatography for enantiomeric separations.

M. Ausborn et al., U.S. patent application publication 2006/0147532 disclose a method for preparing microparticles by dissolving, dispersing or emulsifying an active agent in a biocompatible, biodegradable polymer and an ionic liquid, to form a mixture; and removing the ionic liquid from the mixture, thereby forming microparticles containing the active agent embedded within a polymeric matrix.

ILs have been used for a range of applications, including safer organic reactions (e.g., “greener” Grignard chemistry), analytical chemistry, and materials synthesis. For instance, several studies have described the use of room temperature ILs as polar domains in preparing microemulsions. ILs have also been used as media for the synthesis of functional inorganic nanoparticles and other nanostructures, including gold and platinum nanoparticles, silver and gold nanowires, and cobalt-platinum nanorods.

Z. Li et al., “Synthesis of Single-Crystal Gold Nanosheets of Large Size in Ionic Liquids,” J. Phys. Chem. B 2005, 109, 14445-14448 discloses the preparation of large-size single-crystal gold nanosheets of HAuCl₄ in the ionic liquid 1-butyl-3-methylimidazolium tetrafluoroborate.

M. Antonietti et al., “Ionic Liquids for the Convenient Synthesis of Functional Nanoparticles and Other Inorganic Nanostructures,” Angew. Chem. Int. Ed. 2004, 43, 4988-4992 provides a review of methods that had been used for preparing nanocrystals and nanostructures in ionic liquid solvents.

J. Eastoe et al., “Ionic Liquid-in-Oil Microemulsions,” J. Am. Chem. Soc. 2005, 127, 7302-7303 discloses the formation of ionic liquid-in-oil microemulsions, stabilized with surfactants, to provide microheterogeneous systems for use as reaction and separation media.

Y. Wang, “Synthesis of CoPt Nanorods in Ionic Liquids,” J. Am. Chem. Soc., 2005, 127 (15), 5316-5317 discloses the high-temperature (˜350° C.) synthesis of nanorods, hyperbranched nanorods, and nanoparticles with different CoPt compositions in the ionic liquid 1-butyl-3-methylimidazolium bis(trifluoromethylsulfonyl)imide.

M. Shigeyasu et al., “Formation process and chemical structure analysis of ionic-liquid nanoparticle,” Abstract T09A003, European Aerosol Conference 2007, Salzburg, Austria (Sep. 11, 2007) discloses the formation of ionic-liquid nanoparticles from [C₄mpyrr][NTf₂], 1-butyl-1-methylpyrrolidinium bis(trifluoromethanesulphonyl)imide, by heating to 250° in a furnace, followed by cooling. Further details were given in a later publication, M. Shigeyasu et al., “Production of nanoparticles composed of ionic liquid [C₄ mpyrr][NTf₂] and their chemical identification by diameter analysis and X-ray photoelectron spectroscopy,” Chemical Physics Letters 463 (2008) 373-377 (available online Aug. 22, 2008). The authors of the abstract stated, “On the other hands, recently, ionic liquids of which melting temperatures are extremely low have attracted much attention because of their novelty and specific properties.” Indeed, [C₄ mpyrr][NTf₂] has a melting point of −9° C. See I. Krossing et al., “Why Are Ionic Liquids Liquid? A Simple Explanation Based on Lattice and Solvation Energies,” J. Am. Chem. Soc., 2006, 128 (41), 13427-13434, particularly Table 2 and Scheme 1 on p. 13431. At room temperature, [C₄ mpyrr][NTf₂] is a viscous liquid.

G. Baker et al., “An Analytical View of Ionic Liquids,” Analyst, 2005, 130, 800-808 provides a review of the use of ionic liquids as solvents in analytical chemistry, including for example uses in gas and liquid chromatography, capillary electrophoresis, and others.

T. Ramnial et al., “Phosphonium ionic liquids as reaction media for strong bases,” Chem. Commun., 2005, 325-327 discloses that certain phosphonium ionic liquids are stable in the presence of strong bases, and thus may be used as reaction media for strong bases, for example Grignard reagents.

D. Xiao et al., “Size-Tunable Emission from 1,3-Diphenyl-5-(2-anthryl)-2-pyrazoline Nanoparticles,” J. Am. Chem. Soc., 2003, 125 (22), 6740-6745 discloses the preparation of nanoparticles of 1,3-diphenyl-5-(2-anthryl)-2-pyrazoline ranging in average diameter from 40 to 160 nm by reprecipitation from an acetonitrile solution rapidly injected into water at room temperature.

Conventional work with ILs has focused almost entirely on those whose melting points are below ˜25° C., to take advantage of their beneficial properties in reactions, syntheses, and separations at ambient or near-ambient conditions. There have been very few prior reports describing any practical uses for ionic liquids with melting points above room temperature, 25° C. (sometimes called “frozen” ionic liquids). F. Rutten et al., Angewandte Chemie, International Edition. 2007, 46, 4163-4165 demonstrated rewritable imaging on the surfaces of frozen IL substrates.

Some low-melting point magnetic ionic liquids have been reported, generally containing transition metals, such as high-spin d5 iron (III) in the form of tetrachloro- or tetrabromo-ferrate (III), or gadoliniuum (III), with various counter cations. See, e.g., S. Hayashi et al., “Discovery of a magnetic ionic liquid [bmim]FeCl₄,” Chemistry Letters (2004), 33(12), 1590-1591.

To our knowledge, there have been no prior reports or prior suggestions of preparing solid microparticles or nanoparticles from ionic liquids, those with melting points above 25° C.; as opposed to liquid vesicles or other liquid-phase compositions. To our knowledge there have been no prior reports of any uses for “frozen” ionic liquids, other than Rutten et al.'s report of rewritable imaging on the surfaces of frozen IL substrates.

### SUMMARY OF THE INVENTION

We have discovered novel microparticles and nanoparticles prepared from solid (“frozen”) ionic liquids, as well as methods for making the novel microparticles and nanoparticles. The novel microparticles and nanoparticles have a wide variety of uses. The particle size depends on processing conditions that the user may control, and the properties of the particles are “tunable.” IL microparticles and nanoparticles can be considered “designer particles,” because their properties may be tailored or tuned to meet specific needs, by suitably choosing the cation, the anion, or both. As just one example, their composition may be chosen to make them biodegradable, or to be robust under harsh physiological conditions.

“Frozen” IL nanoparticles have distinct properties from other types of nanoparticles. ILs are broadly tunable by modifying the anionic constituents, the cationic constituents, or both; meaning that many properties may readily be altered, such as melting point, density, viscosity, surface tension, solubility, tensile strength, hydrophobicity, hydrophilicity, rigidity, reactivity, radioactivity, magnetic properties, optical properties, and other physical and chemical properties. IL nanoparticles can thus be designed to optimize one or more properties for particular applications, such as fluorescence, chirality, non-toxicity, biodegradability, photoluminescence, self-assembly, heavy metal scavenging, antiviral or antimicrobial properties. By tuning the properties of the nanoparticles or microparticles, in some cases there will be a correspondingly reduced need for separate chemical activation or loading of active ingredients into the particles. The properties of ILs are sufficiently tunable that they can mimic many of the properties of “conventional” particle types, in addition to providing qualities that are not readily obtained in polymeric, silica, metal, and other types of particles previously known in the art. For example, J. Huang et al., Journal of the American Chemical Society. 2005, 127, 12784 reported a blue-emitting photoluminescent IL, and a proton-conductive IL built around a polyamidoamine (PAMAM) dendrimer core; and A. Boydston et al., Journal of the American Chemical Society. 2007, 129, 14550 reported phase-tunable fluorophores based on benzobis(imidazolium) salts. Frozen ILs may be chosen to be environmentally-friendly or biocompatible. Some examples of high-melting-temperature (“frozen”) ILs are given in Tables 1 and 2 below, and other examples are known in the art.

We have also discovered several methods for making the novel IL microparticles and nanoparticles. One method is based on an oil-in-water emulsion procedure that is rapid, simple, and eliminates the need for an adsorbed surfactant, stabilizer, or toxic organic solvent—one or more of which have traditionally been used in synthesizing prior types of nanoparticles. The use of organic solvents not only presents environmental concerns, but it can also be harmful to the delivery of a drug molecule of interest. It can also be difficult to remove adsorbed surfactants after the synthesis of conventional nanoparticles. In addition, the size of the IL particles can be easily controlled by modifying the preparation conditions.

IL particles can be made with a controlled size and controlled dispersity. Smaller, monodisperse particle sizes can be used, for example, in separations to shorten analysis times and improve separation efficiencies. ILs have very low vapor pressures, rendering them more environmentally friendly than volatile organic solvents in this respect. Some ILs can withstand very high temperatures.

The novel IL nanoparticles may be tailored to replace essentially any of the conventional nanoparticles presently in use. For example, silica nanoparticles have become popular for a number of uses due to their low toxicity. The novel ILs can be tailored to have properties comparable to or better than those of silica nanoparticles.

### BRIEF DESCRIPTION OF THE DRAWINGS

FIGS. 1(a) and 1(b) illustrate schematically the steps involved in two embodiments of the melt-emulsion-quench method for synthesizing nano- and microparticles, without surfactant, and with surfactant, respectively.

FIGS. 2(a) and 2(b) depict scanning electron microscopy images and transmission electron microscopy images, respectively, of the [bm₂Im][PF₆] nanoparticles.

FIG. 3 depicts 5 μm particles prepared using homogenization, followed directly by chilling on ice without sonication.

FIG. 4 depicts the morphology of nanoparticles prepared with an emulsifier.

FIG. 5 depicts schematically a proposed structure for a PAMAM-OH G4 dendrimer, with an imidazolium-based siloxy-terminated IL.

FIG. 6(a) depicts schematically the electrospinning apparatus used to produce electrospun nanofibers. FIG. 6(b) depicts an SEM micrograph of the resulting electrospun nanofibers.

FIGS. 7(a), (b), 8(a), (b), 9(a), (b), and 10(a), (b) depict electron micrographs of several inkjet dispersal preparations.

FIGS. 11(a) and (d) depict, respectively, absorbance and fluorescence emission spectra of ionic liquid nanoparticles prepared from an NIR dye.

### MODES FOR PRACTICING THE INVENTION

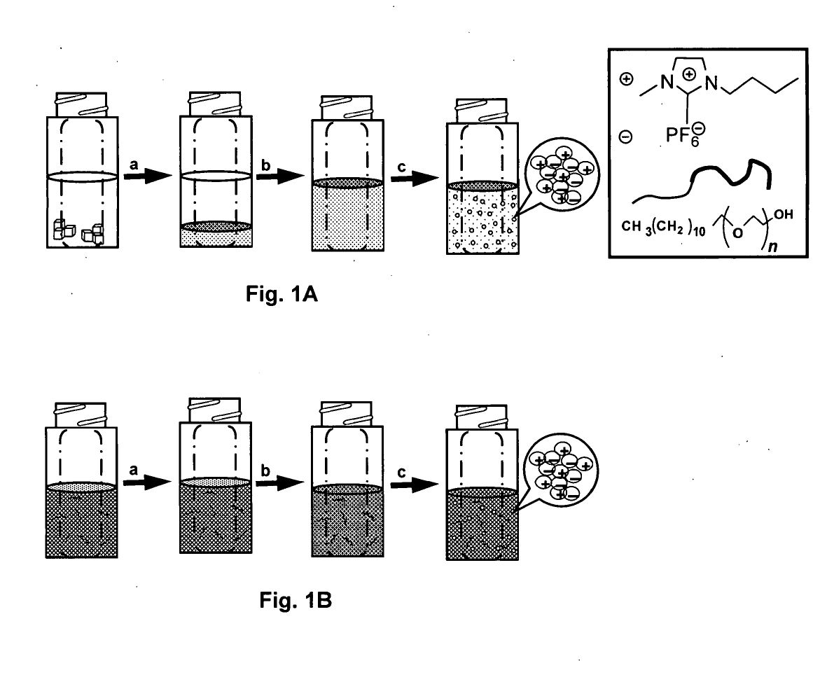

The starting material used in one prototype embodiment was solid 1-butyl-2,3-dimethylimidazolium hexafluorophosphate ([bm₂Im][PF₆]), an IL with a melting point of 42° C. IL particles were prepared by two alternative methods, illustrated schematically in FIGS. 1(a) and 1(b). The first method employed the melting and then the subsequent oil/water (“o/w”) dispersion of the liquid-phase [bm₂Im][PF₆] into water at a temperature well above the IL's melting point, followed by rapid cooling to produce discrete, solid IL nanoparticles. The second method is broadly similar, but also employed an emulsifier, the nonionic surfactant polyoxyethylene (23) lauryl ether (Brij® 35). When we employed the lipophilic dye Nile Red as a visualization aid, the dye did not appreciably color the aqueous component, but was instead incorporated almost entirely into the intermediate o/w microemulsion, and thence into the final IL nanoparticles. Incorporation of Nile Red allowed easy visualization of the IL nanoparticles, and showed that IL nanoparticles are well-suited to entrap various materials, e.g., drugs, magnetic compounds, or sensory agents where it is desirable to do so.

FIGS. 1(a) and 1(b) illustrate schematically the steps involved in embodiments of the melt-emulsion-quench method for synthesizing nano- and microparticles without surfactant (FIG. 1(a)) and with surfactant (FIG. 1(b)). In FIG. 1(a), the first step (a) depicts the melting of the salt in a hot water bath, while dropwise addition of molten salt to a surfactant solution is performed in the process of FIG. 1(b). The subsequent steps are homogenization and probe sonication (b), followed by rapid quenching in an ice bath to solidify (“freeze”) the particles (c). Alternatively, reverse micelles may be used in this process.

Another method, useful for example to manufacture solid nanoparticles containing active pharmaceutical ingredients (APIs) involves evaporation from an emulsion. An emulsification step, for example high shear mixing with a rotor-stator mixer, or high pressure homogenization, or sonication, first produces an o/w or a w/o emulsion. Particles are then formed by solvent evaporation via increased heat, reduced pressure, or both. The melt-emulsion-quench process is well suited both for low-melting ILs as well as for those with melting points up to 200° C., or even higher. This method is far more energy-efficient than other methods that have previously been used to produce API-containing (non-IL) nanoparticles. The ILs themselves can have antimicrobial activity, particularly those in which the anion component of the salt is itself an API. For antimicrobial ionic liquids, one might include, for example, a silver-ion-containing complex IL such as those reported earlier by Dai et al. in J. Electrochem. Soc. 2006, 153, J9 {e.g., [Ag(RNH₂)²⁺][Tf₂N⁻]}, where RNH₂ is an alkylamine. Antimicrobial cations include, for example, those derived from chlorophenol, from octanaminium, from thymol, or from benzyl ammonium, quaternary ammonium, and others that are known in the art, preferably those that have previously been EPA-approved for antibacterial activity against pathogens. Antiviral cations include those derived from toluamide, from amylphenol, from chlorocyanurate, from chlorotriazinetrione, and others that are known in the art, preferably those that have previously been EPA-approved for activity against viruses such as HIV and Hepatitis.

The preparations can be conducted under mild conditions, for example a melt-emulsion-quench technique employing the molten IL itself as the oil phase of an o/w microemulsion. No costly or specialized equipment is necessarily required, nor (in many cases) need an organic solvent be used at any stage of the process. The particle preparation process can be readily scaled up to grams, kilograms, or even larger. Particle geometry, dimensions, and composition can be controlled by varying reaction conditions such as temperature, pressure, sonication conditions (if any), surfactant choice (if any), selection of IL building blocks, and emulsion type. Additional layers may optionally be added in multiple emulsions, such as oil-in-water-in-oil (o/w/o) systems. Some ILs are amenable to templating, and can optionally be templated using porous materials, polymer aggregates, dendrimers, other organized media, or lithography techniques. Also, “frozen IL” nanostructures may be made via techniques such as electrospinning (fibers) or electrospray (particles).

The novel frozen ILs may be used in a variety of areas, including biomedical imaging, displays, “intelligent” inks, actuators, sensory devices, fuel cells, self-healing materials, separations, batteries, switches, fabrics, modified electrodes, antimicrobial surfaces, and “lab-on-chip” constructs.

### Example 1

We used solid, amorphous, millimeter-sized granules of [bm_{2l Im][PF}₆] as a starting material in a melt-emulsion-quench process, without surfactant (Method 1), to produce controlled particle sizes of nanometer or micron dimensions, depending on the conditions. In a typical preparation, 25 mg of solid [bm₂Im][PF₆] was gently rinsed several times in ultrapure water (18.2 MΩ/cm), and then was added to 8 mL of ultrapure water in a 20 mL scintillation vial. The sealed vial was heated to 70° C. in a water bath until the [bm₂Im][PF₆] melted to form a clear, dense liquid phase. The mixture was then homogenized using a commercial homogenizer (PowerGen 125, Fisher Scientific) at 30,000 rpm for 10 min, while the sample was maintained at 70° C. in the water bath. The mixture was then sonicated with a probe ultrasound processor (model CV330, Sonics and Materials Inc., Newton, Conn., USA) at 35% intensity (i.e., 35% of a maximum 300W power output) for 10 min. (The sonication frequency and power affected the size of the particles, although not strongly. We observed that lower frequency sonication tended to produce more uniform particles, and higher power tends to produce smaller particles. However, these effects were not very pronounced; we did not observe the size to change more than ˜20 nm, nor the uniformity to vary more than ˜2-3% of the standard deviation, under the conditions we tested.) Following sonication, the mixture was immediately placed into an ice-water bath to rapidly reduce the temperature below the melting point of the IL. The resulting nanoparticles, suspended in the aqueous phase, were washed by ultrafiltration (Millipore) three times to remove soluble species.

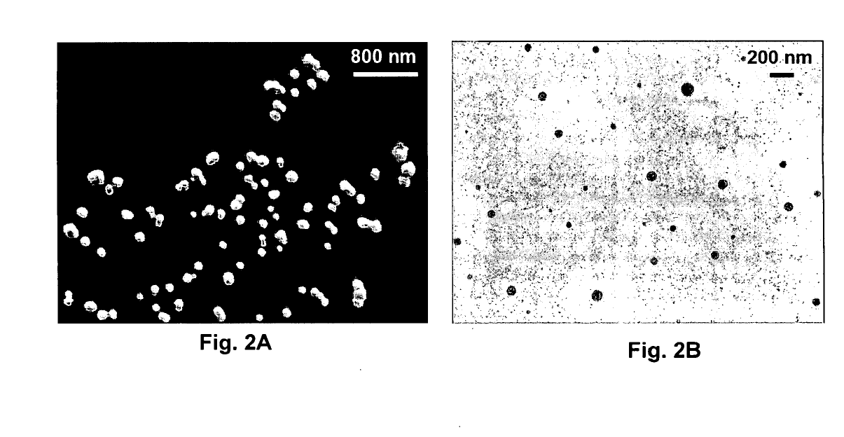

Scanning electron microscopy (SEM) images of the [bm₂Im][PF₆] nanoparticles showed that they were generally spherical, with a diameter of 90±32 nm, as shown in FIG. 2a. They formed a single layer on the transmission electron microscopy (TEM) grid surface, with minimal interparticle aggregation. TEM also showed generally spherical particles, with a diameter measured as 88±34 nm. We found that chilling the o/w emulsion on ice helped minimize aggregation of particles, by promoting swift IL solidification, and by inhibiting the merging of isolated droplets prior to freezing. The average nanoparticle diameter measured by SEM and TEM imaging was confirmed by dynamic light scattering (DLS). The DLS polydispersity index (PDI) of the as-prepared [bm₂Im][PF₆] nanoparticles, an estimate of the size distribution width, was as low as 0.105.

### Examples 2-4

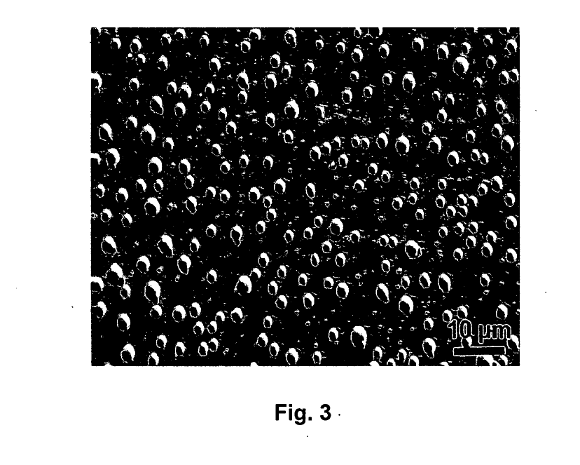

We found that particle size could be varied by altering the conditions employed in preparation: temperature; homogenization speed and duration; and sonication intensity, duration, and pulse interval sequence. For example, following a protocol as otherwise described for Example 1, a 30 second homogenization, followed directly by chilling on ice without sonication, produced particles about 5 μm in diameter (see FIG. 3); a 10 minute sonication produced particles about 250 nm in diameter; and a 20 minute sonication produced nanoparticles less than 100 nm in diameter.

### Example 5

By changing the solvent, a wider range of operating temperatures may be used at atmospheric pressure than is feasible with water alone. Using water as a solvent restricts one to using ILs with melting points below 100° C. (unless the system is operated at higher pressures to increase the boiling point of water, which is of course possible but makes the synthesis little less convenient). In addition to facilitating the use of higher-melting ILs, employing non-aqueous solvents or solvent-water uniphasic or biphasic mixtures can also help fine-tune particle size and shape, particularly by varying the surface tension of the solvent. Higher-boiling solvents include, for example, 1-octadecene, phenyl ether, ethylene glycol and polyethylene glycol. Another example is glycerol, which has a high viscosity (934 mPa-s or cp at 25° C.), and a high boiling point (290° C.). In addition, or in the alternative, hydrothermal vessels may be used (e.g., with microwave irradiation), as their use can extend the temperature range even for water-containing systems because of the autogenous pressure that is generated (e.g., ionothermal synthesis).

### Example 6

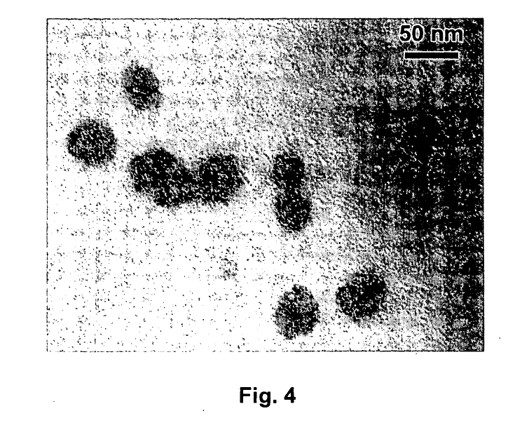

Emulsifying agents generally orient preferentially at the interface between the oil (e.g., [bm₂Im] [PF₆] or other IL) and water phases of the droplets, and thus act to inhibit coalescence. Nanoparticles synthesized with Brij® 35 as an emulsifying agent (Method 2) yielded more monodisperse nanoparticles. For example, we melted 25 mg of [bm₂Im][PF₆] at 70° C., and added it dropwise to a scintillation vial containing 1.0 wt % Brij® 35 in 8 mL of hot, ultrapure water, followed by a 10-minute homogenization, and then was treated as otherwise described in Example 1. This process yielded nanoparticles with a diameter of 45±7 nm. Although these nanoparticles were generally quite uniform, their morphologies appeared to be more irregular and less nearly spherical than those prepared without an emulsifier, as can be seen in the TEM micrograph shown in FIG. 4. The Brij® 35 surfactant apparently provided a protective boundary which preserved particle integrity. In some cases, a surfactant can also provide a convenient route for functionalizing the nanoparticle surface, enhancing the utility of IL nanoparticles. However, in other applications a surfactant layer would be undesirable.

### Example 7

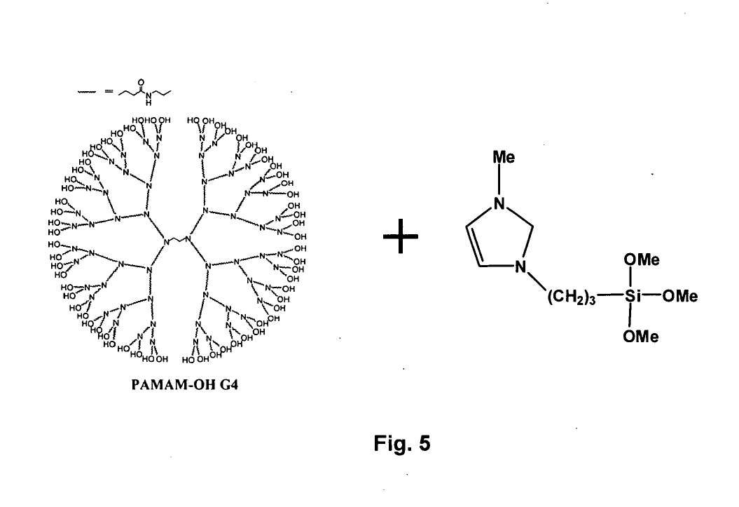

IL particles in accordance with the present invention may optionally be prepared using a dendritic template. For example, PAMAM-OH dendrimers (e.g., any of generations 1 through 12) may be used as nucleating templates for producing IL nanoparticles and microparticles. A siloxy-terminal IL will covalently bind to the hydroxyl terminals of the dendrimer to generate IL particles whose sizes can be controlled based on the generation of dendrimer used, the concentration of the IL in the reaction medium, the time allowed for nucleation, the stirring speed, probe sonication time and intensity, and the pH of the preparation medium. The reaction proceeds at acidic pH, preferably triggered by the addition of a mineral acid such as hydrochloric acid. Without wishing to be bound by this hypothesis, we expect that the range of sizes that may be obtained using a dendritic template should be ˜1 nm-˜500 μm. Somewhat similar approaches have previously been reported for preparing “conventional” silica nanoparticles using dendritic templates. See, e.g., S. Miller et al., Rapid and efficient enzyme encapsulation in a dendrimer silica nanocomposite. Macromolecular Bioscience (2006), 6(10), 839-845. FIG. 5 depicts schematically the PAMAM-OH G4 dendrimer, with an imidazolium-based siloxy-terminated IL.

### Example 8

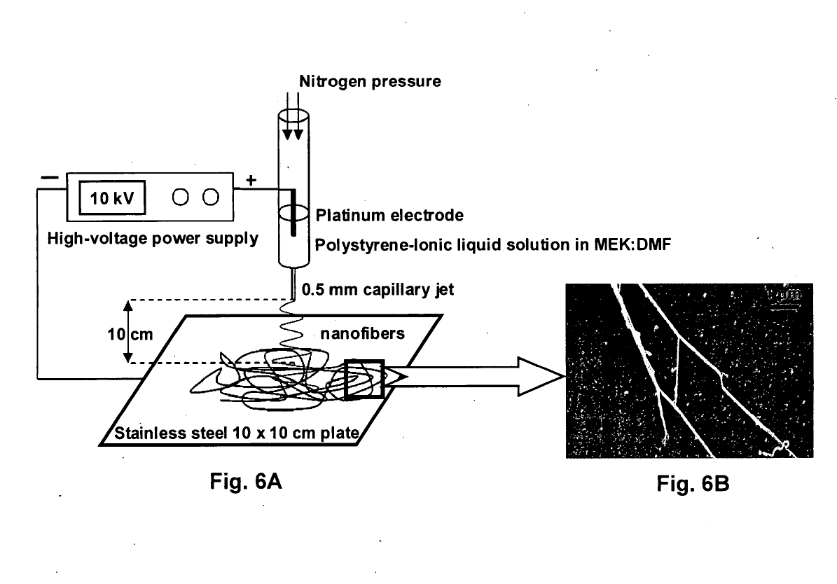

Ionic liquid nanofibers were synthesized using a modification of the electrospinning method of H. Fong et al., Elastomeric nanofibers of styrene-butadiene-styrene triblock copolymer. J. Polym. Sci., Part B: Polym. Phys. 1999, 37, 3488-3493. Due to their low tensile strength, pure ionic liquids did not successfully spin into fibers in our initial attempts. Therefore, a 50:50 (w/w) blend of 1-butyl-2,3-methylimidazolium hexafluorophosphate and polystyrene (100 kDal) was dissolved (0.3 g/ml) at room temperature in a mixture of methyl ethyl ketone (MEK) and N,N-dimethylformamide (DMF) (1:1 v/v) containing 1 wt % lithium chloride (LiCl). Then 1 mL of the solution was loaded into a 3 ml glass tube with an opening at the bottom connected to a silica capillary having a 0.25 mm inner diameter. The top of the glass tube was connected to a pressure-controlled nitrogen cylinder. Inside the tube, a platinum positive electrode was in contact with the solution, while 10 cm under the tube was placed a stainless steel plate used as the negative electrode. Electrospinning was initiated by gradually increasing the potential difference from 0 to 10 kV. Fibers were collected on glass slides or stainless steel mesh, and were then stored in solvent for further analysis. The resulting nanofibers typically had diameters from ˜40 to ˜300 nm, depending on the voltage and nitrogen pressure employed. FIG. 6(a) depicts schematically the electrospinning apparatus used in this experiment, and FIG. 6(b) depicts an SEM micrograph of the resulting electrospun nanofibers. In an alternative embodiment, some ILs have sufficient tensile strength to be spun in a pure state. Likewise, electrospray may be used to make ionic liquid particles or composite materials, by modification of methods that have been used, for example, to make cellulose nanofibers and particles. See, e.g., Chem. Lett. 2008, 37, 114.

### Example 9

Examples 1-6 described the production of water-insoluble nanoparticles by quenching in an aqueous bath. The same approach may be used to produce water-soluble microparticles and nanoparticles, with quenching instead occurring in a hydrophobic solvent in which the ILs are insoluble.

### Example 10

Examples of ILs that may be used in the present invention, and examples of their applications are given in Tables 1 and 2. Other high-melting-temperature ILs known in the art may also be used, in addition to the listed examples.

TABLE 1
Examples of Ionic Liquids with melting points 25-100° C.

| | Example | MP (° C.) | Examples of Applications |
|---|---|---|---|
| Imidazolium- | 1,3-Dimethylimidazolium | 43 | Carriers for biomolecules or analytical |
| Based | trifluoromethanesulfonate | | materials; or these ILs may be doped with dyes |
| | 1-Ethyl-3-methylimidazolium | 88 | for fluorescent analyses. |
| | chloride | | |
| | 1-Ethyl-3-methylimidazolium | 65 | |
| | bromide | | |
| | 1-Butyl-3-methylimidazolium | 73 | |
| | chloride | | |
| | 1-Ethyl-3-methylimidazolium | 56 | |
| | tosylate | | |
| Pyridinium- | N-Butyl-3,4-dimethylpyridinium | 72 | |
| Based | chloride | | |
| | N-Butyl-4-methylpyridinium | 44 | |
| | hexafluorophosphate | | |
| | N-Butylpyridinium | 75 | |
| | hexafluorophosphate | | |
| Ammonium- | Methyltrioctylammonium triflate | 56 | |
| based | Tetraethylammonium tris | 97 | |
| | (pentafluoroethyl)trifluorophosphate | | |
| | Tetrabutylammonium | 92 | |
| | bis(trifluoromethylsulfonyl)imide | | |
| Pyrrolidinium- | 1-Butyl-1-methylpyrrolidinium | 55 | |
| based | bis[oxalato(2-)] bromide | | |
| | 1-Butyl-1-methylpyrrolidinium | 31 | |
| | trifluoroacetate | | |
| | 1-Butyl-1-methylpyrrolidinium | 85 | |
| | hexafluorophosphate | | |
| Phosphonium- | Trihexyltetradecylphosphonium | 25 | |
| Based | tetrafluoroborate | | |
| Amino acid-based | Tetrabutyl ammonium alanate | 76 | Protein-interaction detection, drug delivery |
| | Alanine methyl ester lactate | 38 | carriers, antibody testing, affinity testing, and |
| | Alanine butyl ester tetrafluoroborate | | protein separations. |
| Fluorescent and | Rhodanmine B bis (trifluoromethane) | 80 | Imaging of cells, analytical quantification of |
| absorption dye- | sulfonimide | | free radicals, detection of pathogens in food |
| based | CrystViolet hexafluorophosphate | 60 | products, and other areas in which “quantum |
| | BasicYellow hexafluorophosphate | 85 | dots” (which are generally toxic) have been |
| | Methylviolet2B bis | 48 | used. |
| | (trifluoromethane) sulfonimide | | |
| | MalachiteGreen hexafluorophosphate | 60 | |
| Near infra-red dyes | 1-Butyl-2-(2-{3-[2-(1-butyl-3,3- | 52 | In vivo medical imaging for cancer detection, |
| | dimethyl-1,3-dihydro-indol-2- | | viral identification, and other diagnostic |
| | ylidene)-ethylidene]-2-chloro- | | applications. |
| | cyclohex-1-enyl}-vinyl)-3,3- | | |
| | dimethyl-3H-indolium | | |
| | bis(pentafluoroethylsulfonyl)imide | | |
| | 1-Butyl-2-(2-{3-[2-(1-butyl-3,3- | 87 | |
| | dimethyl-1,3-dihydro-indol-2- | | |
| | ylidene)-ethylidene]-2-chloro- | | |
| | cyclohex-1-enyl}-vinyl)-3,3- | | |
| | dimethyl-3H-indolium tetraphenyl | | |
| | borate | | |
| | 1-Butyl-2-(2-{3-[2-(1-butyl-3,3- | 82 | |
| | dimethyl-1,3-dihydro-indol-2- | | |
| | ylidene)-ethylidene]-2-chloro- | | |
| | cyclohex-1-enyl}-vinyl)-3,3- | | |
| | dimethyl-3H-indolium 3,5- | | |
| | bis(trifluoromethyl)phenyltrifluoroborate | | |
| | 1-Butyl-2-(2-{3-[2-(1-butyl-3,3- | 80 | |
| | dimethyl-1,3-dihydro-indol-2- | | |
| | ylidene)-ethylidene]-2-chloro- | | |
| | cyclohex-1-enyl}-vinyl)-3,3- | | |
| | dimethyl-3H-indolium 4- | | |
| | (trifluoromethyl) | | |
| | phenyltrifluoroborate | | |
| | 1,3,3-Trimethyl-2-[7-(1,3,3- | 98 | |
| | trimethyl-1,3-dihydro-indol-2- | | |
| | ylidene)-hepta-1,3,5-trienyl]-3H- | | |
| | indolium tetraphenyl borate | | |
| | 1,3,3-Trimethyl-2-[7-(1,3,3- | 99 | |
| | trimethyl-1,3-dihydro-indol-2- | | |
| | ylidene)-hepta-1,3,5-trienyl]-3H- | | |
| | indolium 4-(trifluoromethyl) | | |
| | phenyltrifluoroborate | | |
| | 1,3,3-Trimethyl-2-[7-(1,3,3- | 72 | |
| | trimethyl-1,3-dihydro-indol-2- | | |
| | ylidene)-hepta-1,3,5-trienyl]-3H- | | |
| | indolium tetrakis[3,5-bis(1,1,1,3,3,3- | | |
| | hexafluoro-2-methoxy-2- | | |
| | propyl)phenyl]borate | | |
| | 2-(2-{2-Chloro-3-[2-(1,3,3-trimethyl- | 89 | |
| | 1,3-dihydro-indol-2-ylidene)- | | |
| | ethylidene]-cyclohex-1-enyl}-vinyl)- | | |
| | 1,3,3-trimethyl-3H-indolium bis | | |
| | (trifluoromethane) sulfonimide | | |
| | 2-[2-[2-Chloro-3-[2-(1,3-dihydro- | 85 | |
| | 1,3,3-trimethyl-2H-indol-2-ylidene)- | | |
| | ethylidene]-1-cyclopenten-1-yl- | | |
| | ethenyl]-1,3,3-trimethyl-3H-indolium | | |
| | hexafluoroposphate | | |
| Vitamin-based | Vitamin B4 lactate | 100 | Carriers for drug delivery featuring intrinsic |
| | Nicotinamide adenine dinucleotide | 40 | non-toxicity and high biodegradability |
| | lactate | | |
| | Riboflavin 5′-adenosine diphosphate | 40 | |
| | lactate | | |
| Anti-bacterial/viral | Amantadine bis (trifluoromethane) | 95 | In vivo use in implants, surgical equipment, |
| compound-based | sulfonimide | | and household items |

See generally also:
(1) the Ionic Liquid Data Bank, NIST Standard Reference Database #147, currently available online at ilthermo.boulder.nist.gov;
(2) H. Ohno et al., Accounts of Chemical Research. 2007, 40, 1122; and
(3) M. Patil et al., Tetrahedron. 2007, 63, 12702.

TABLE 2
Examples of Ionic Liquids with melting points 100-200° C.

| | Example | MP(° C.) | Application |
|---|---|---|---|
| Imidazolium- | 1-Dodecyl-3-methylimidazolium | 134 | These frozen ILs have not previously found |
| based | chloride | | any general, reported utility, other than as |
| | 1-Ethyl-2,3-dimethylimidazolium | 138 | solvents for high temperature organic |
| | bromide | | synthesis. As microparticles or nanoparticles, |
| | 1-Ethyl-2,3-dimethylimidazolium | 110 | these ILs may, for example, be used as a |
| | trifluoromethanesulfonate | | substitute for silica as carriers for |
| Pyridinium- | N-Butylpyridinium bromide | 105 | biomolecules or analytical materials, or they |
| based | N-Butyl-3-methylpyridinium chloride | 117 | could be doped with dyes for fluorescent |
| | N-Ethylpyridinium chloride | 119 | analyses. |
| Ammonium- | Tetramethylammonium bis[oxalato(2-)] | 130 | |
| based | bromide | | |
| | Tetramethylammonium tris | 115 | |
| | (pentafluoroethyl)trifluorophosphate | | |
| | Tetramethylammonium | 135 | |
| | bis(trifluoromethanesulfonyl)imide | | |
| Pyrrolidinium- | 1,1-Dimethylpyrrolidinium | 107 | |
| based | tris(pentafluoroethyl)trifluorophosphate | | |
| | 1-Butyl-1-methylpyrrolidinium | 147 | |
| | tetrafluoroborate | | |
| Amino acid-based | Alanine butyl ester nitrate | 104 | Protein-interaction detection, drug delivery |
| | Alanine butyl ester lactate | 114 | carriers, antibody testing, affinity testing, and |
| | | | protein separations. |
| Fluorescent dye- | Rhodamine 6G nitrate | 126 | Imaging of cells, analytical quantification of |
| based | Crystal Violet | 170 | free radicals, detection of pathogens in food |
| | bis(trifluoromethanesulfonyl)imide | | products, and other areas where “quantum |
| | Thioflav | 169 | dots” (which are generally toxic) have been |
| | bis(trifluoromethanesulfonyl)imide | | used. |
| | BasicYellow | 127 | |
| | bis(trifluoromethanesulfonyl)imide | | |
| Near infra-red dyes | 1-Butyl-2-(2-{3-[2-(1-butyl-3,3- | >120 | In vivo medical imaging for cancer detection, |
| | dimethyl-1,3-dihydro-indol-2- | | viral identification, and other diagnostic |
| | ylidene)-ethylidene]-2-chloro- | | applications. |
| | cyclohex-1-enyl}-vinyl)-3,3- | | |
| | dimethyl-3H-indolium | | |
| | bis(trifluoromethanesulfonyl)imide | | |
| | 1-Butyl-2-(2-{3-[2-(1-butyl-3,3- | >120 | |
| | dimethyl-1,3-dihydro-indol-2- | | |
| | ylidene)-ethylidene]-2-chloro- | | |
| | cyclohex-1-enyl}-vinyl)-3,3- | | |
| | dimethyl-3H-indolium | | |
| | trifluorophenylborate | | |
| | 1,3,3-Trimethyl-2-[7-(1,3,3- | >120 | |
| | trimethyl-1,3-dihydro-indol-2- | | |
| | ylidene)-hepta-1,3,5-trienyl]-3H- | | |
| | indolium | | |
| | bis(pentafluoroethylsulfonyl)imide | | |
| | 1,3,3-Trimethyl-2-[7-(1,3,3- | >120 | |
| | trimethyl-1,3-dihydro-indol-2- | | |
| | ylidene)-hepta-1,3,5-trienyl]-3H- | | |
| | indolium | | |
| | bis(trifluoromethanesulfonyl)imide | | |
| | 1,3,3-Trimethyl-2-[7-(1,3,3- | >120 | |
| | trimethyl-1,3-dihydro-indol-2- | | |
| | ylidene)-hepta-1,3,5-trienyl]-3H- | | |
| | indolium 3,5- | | |
| | bis(trifluoromethyl)phenyltrifluoroborate | | |
| | 1,3,3-Trimethyl-2-[7-(1,3,3- | >120 | |
| | trimethyl-1,3-dihydro-indol-2- | | |
| | ylidene)-hepta-1,3,5-trienyl]-3H- | | |
| | indolium tetrafluoroborate | | |
| | 2-(2-{2-Chloro-3-[2-(1,3,3-trimethyl- | >100 | |
| | 1,3-dihydro-indol-2-ylidene)- | | |
| | ethylidene]-cyclohex-1-enyl}-vinyl)- | | |
| | 1,3,3-trimethyl-3H-indolium | | |
| | bis(pentafluoroethylsulfonyl)imide | | |
| Vitamin-based | 1-Butyl-2-(2-{3-[2-(1-butyl-3,3- | 140 | Carriers for drug delivery featuring intrinsic |
| | dimethyl-1,3-dihydro-indol-2- | | non-toxicity and high biodegradability. |
| | ylidene)-ethylidene]-2-chloro- | | |
| | cyclohex-1-enyl}-vinyl)-3,3- | | |
| | dimethyl-3H-indolium | | |
| | bis(trifluoromethanesulfonyl)imide | | |
| Anti-bacterial/viral | 1-Butyl-2-(2-{3-[2-(1-butyl-3,3- | 180 | Use in implants in vivo, surgical equipment, |
| compound-based | dimethyl-1,3-dihydro-indol-2- | | and household items |
| | ylidene)-ethylidene]-2-chloro- | | |
| | cyclohex-1-enyl}-vinyl)-3,3- | | |
| | dimethyl-3H-indolium | | |
| | trifluorophenylborate | | |

Additional ionic liquids that might be used in one or more of the above applications include, for example: Rhod6G NO₃, CrystViol NTf₂, Thioflav NTf₂, BasicYellow NTf₂, VitB₄ PF₆ and Tetracycline NTf₂.

### Examples 11-13

Frozen IL particles may be prepared to contain cationic or anionic active components, e.g., fluorophores, antibacterial compounds, ligand-toxin conjugates, etc., in at least two different ways: (1) The active component may itself be the anion or cation component of the ionic liquid, or (2) the active component is incorporated into a frozen IL with different anionic and cationic components, preferably assisted by a non-ionic surfactant. The particles produced by the two methods will generally share many similar properties; a principal difference is that particles produced using method (1) in many cases can more readily be made without surfactant, and thus may be preferred for some in vivo applications.

Using a surfactant enhances control over size, and often results in smaller particles. An example using method (1) with 1-butyl-2,3-dimethylimidazolium hexafluorophosphate gave particles 90 nm in diameter, while using method (2) with the same IL produced particles having a 45 nm diameter.

An example where the active ingredient is itself a component of the ionic liquid is Rhodamine B NTf₂. Rhodamine B is a cent dye. The fluorescent properties of Rhodamine B are carried into the microparticles and nanoparticles, which may be used in applications such as medical imaging and other applications where semi-conductor “quantum dots” (which are often toxic) have previously been used.

### Example 14

### Using Aerosolization to Prepare IL Microparticles and Nanoparticles

Aerosol techniques have previously been used to form silica or metal nanoparticles. These techniques can produce particles over a wide range of sizes, but with a narrow size range for a given selected size. We have modified prior aerosol techniques as an alternative to make microparticles and nanoparticles from the ionic liquid 1-butyl-2,3-dimethylimidazolium hexafluorophosphate (bm₂Im.PF₆). In different experiments, we have successfully made particles with mean sizes ranging from 20 nm to 10 μm. The size and size uniformity of the prepared particles are functions of the air flow rate, the ionic liquid concentration in the reservoir, the use of a size-selector, and the furnace temperature.

In one embodiment, pressurized air flow proceeded through several components in the following order (as explained further below): (A) an air filter, (B) a constant-output atomizer with a reservoir containing a solution of the ionic liquid, (C) a silica gel-based dryer, (D) an electrostatic classifier to sort droplets by size, (E) a tube furnace (Tₘₐₓ=1100° C.), and (F) a flow direction valve, which could direct the output of the furnace to either: (G-1) a filter holder with a 1 μm Teflon filter, or (G-2) an ultrafine condensation particle counter.

Using a stock solution of ionic liquid in methanol, concentration 1 μM to 10 mM, the solution was aerosolized using purified air from a Zero-Air™ generator. The aerosolized solution was then sent to a Differential Mobility Analyzer (DMA), which permitted only allow selected sizes of nanodroplets to pass. The size-selected droplets were then sent through a tube furnace at a selected temperature in the range 50-400° C. Dry particles exiting the furnace were collected on a 1 μm teflon filter. Size measurements were taken approximately every hour using a Scanning Mobility Particle Sizer (SMPS). In addition to the SMPS, Electron Microscopy and Dynamic Light Scattering were used to measure the average particle size, shape, and polydispersity of the particles.

Particles in the range ˜20 nm to ˜10 μm have been successfully prepared by the aerosol preparation technique. We observed that the concentration of ionic liquid in the stock reservoir strongly affected the particle size, while the temperature of the tube furnace strongly affected the polydispersity of the particles. The lowest polydispersity was observed at the highest temperature tested, 400° C. As measured by an ultrafine condensation particle counter, the particles had an average diameter of 94±37 nm. As measured by dynamic light scattering the particles had an average diameter of 118˜58 nm, with the lowest polydispersity index we observed in this set of experiments, 0.156.

### Examples 15-18

### Using Inkjet Microdispensing to Prepare IL Microparticles and Nanoparticles

Inkjet microdispensing techniques have recently been reported for producing conventional uniform-sized nano- and microparticles. See Patel et al., Asia-Pac. J. Chem. Eng. 2007, 2, 415-430. Uniform-sized droplets are pumped through a nozzle by a piezo-electric actuator. Using a 100 μm nozzle, we produced uniform IL particles from 50 nm to 500 μm.

A prototype of such a microdispensing system comprised: (A) a microdrop controller, (B) a nozzle, (C) an ionic liquid solution reservoir, (D) a glass container containing dispersant under stirring, and (E) an adjustable-speed magnetic stirring plate. In one set of experiments, tetrabutylammonium bis(trifluoromethylsulfonyl)imide (TBA), an ionic liquid with a melting point of 82° C., was first dissolved in a polar solvent such as methanol, ethanol, iso-propanol, or acetonitrile, and the solution was then dispensed into water, in which TBA is insoluble. Dispensing into water causes the precipitation of nano- or micro-particles. Among the factors that can be varied to alter the particles' size, shape, and uniformity are the frequency of the piezoelectric, the solvent, the concentration of TBA in the solution, additives in the organic solvent or in the water, the distance between the nozzle and the surface of the water, and the use of a different “non-solvent” liquid other than pure water. Particles have been prepared to date from below 100 nm to a few micrometers with this technique.

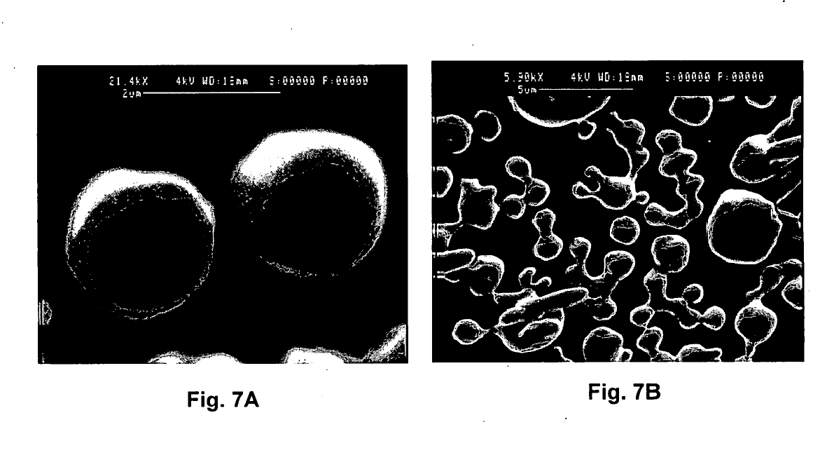

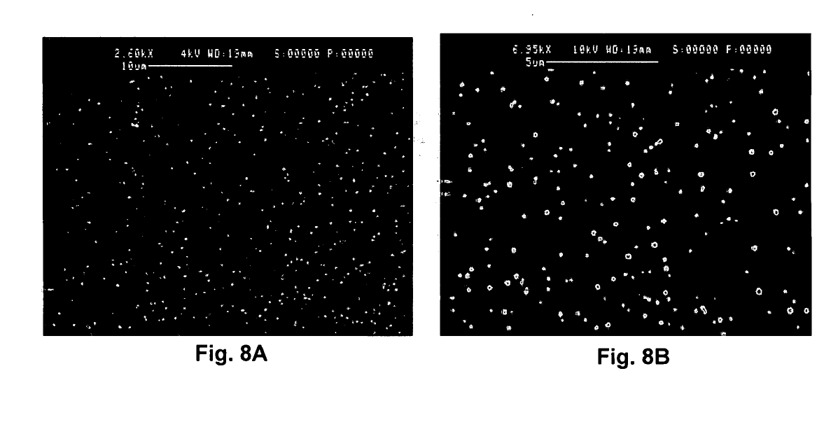

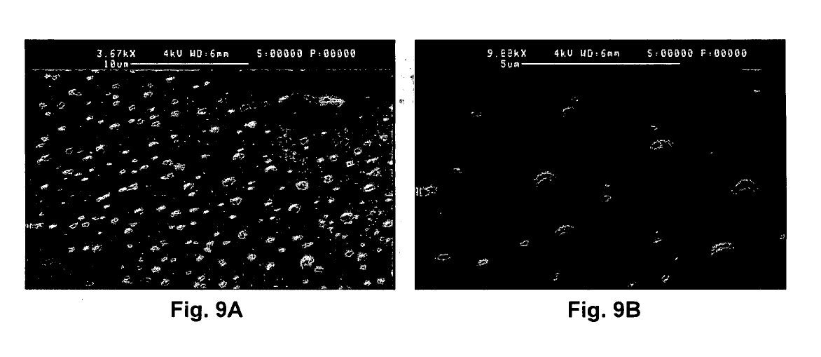

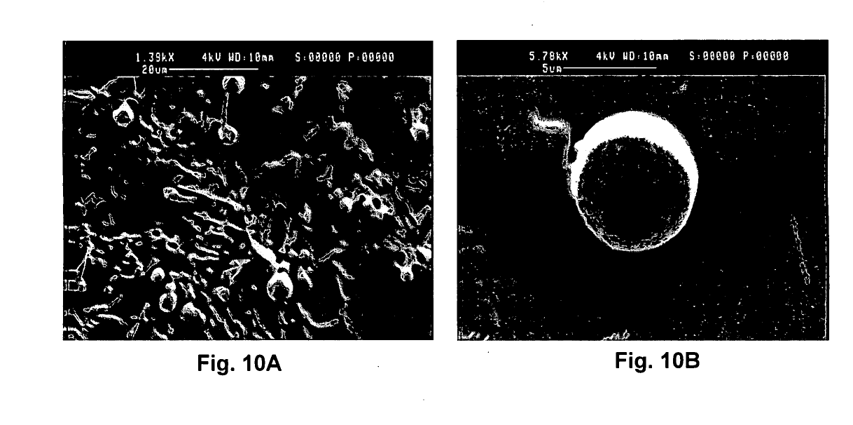

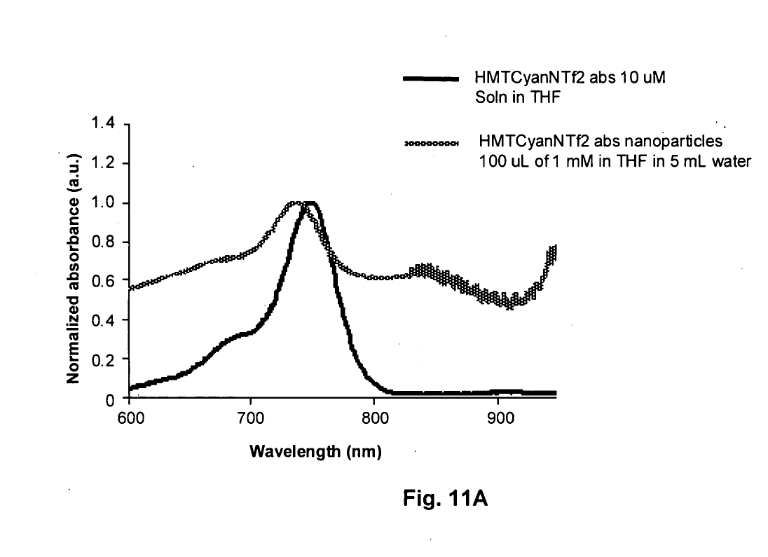

Electron micrographs of several inkjet dispersal preparations are depicted in FIGS. 7(a), (b), 8(a), (b), 9(a), (b), and 10(a), (b). FIGS. 7(a), 7(b) depict SEM images of the particles produced from 10 mM tetrabutylammonium (TBA) dissolved in ethanol (EtOH), and dispensed in H₂O at 1500 Hz. FIGS. 8(a), (b) depict SEM images of particles produced from 10 mM TBA dissolved in EtOH, and dispensed in H₂O at 250 Hz. FIGS. 9(a), (b) depict SEM images of particles produced from 2 mM TBA dissolved in acetonitrile, and dispensed in H₂O at 1500 Hz. FIGS. 10(a), (b) depict SEM images of particles produced from 50 mM TBA in acetonitrile, and dispensed in H₂O at 1000 Hz.

### Examples 19 and 20

### Using Re-Precipitation to Prepare IL Microparticles and Nanoparticles from Near Infrared Dye Ionic Liquids

We have prepared near infrared (NIR) fluorescent “frozen ionic liquid” nanoparticles using a simple reprecipitation method. The NIR ionic liquids were synthesized using an anion exchange metathesis reaction between cationic dye halides, such as iodide or chloride, and anions such as bis(trifluoromethane) sulfonimide and hexafluorophosphate. A solution of the ionic liquid was then dispersed into a “non-solvent” dispersant, such as water, and “frozen ionic liquid” particles then precipitated. The size of the nanoparticles was determined by dynamic light scattering, as well as by electron and optical microscopy. The results showed that the resulting particle diameters were between 50-400 nm. The optical properties of the nanoparticles were studied by UV-visible absorption and fluorescence spectroscopy. The NIR dye ionic liquids that we studied in prototype experiments absorbed in the range 740-800 nm, with emission in the range 750-850 nm.

The absorbance and emission properties of these NIR ionic liquid nanoparticles make them well-suited for biomedical imaging, because body tissues do not absorb strongly at NIR wavelengths. NIR dye nanoparticles derived from ionic liquids have particularly interesting properties, owing to their negligible vapor pressure, and the capability to tune their properties, as previously discussed. In addition, the compositions of the dyes may be chosen to make them biodegradable.

Prototype particles were prepared via a simple, additive-free reprecipitation method that was generally similar to methods that have previously been used to prepare conventional organic nanoparticles. In a typical preparation, 100 μL of a 0.1-2.0 mM solution of the ionic liquid dye in a water-miscible solvent, such as THF, acetonitrile or ethanol, was rapidly injected into 5-10 mL triply deionized water with vigorous stirring or probe sonication. Prior to injection, the ionic liquid dye solutions and water were filtered with 0.2 μm membranes. A modified approach used a greater volume of a solution with a lower concentration of the dye, in an otherwise similar process—for example a 0.02 mM solution of the dye in a water-miscible solvent such as THF, mixed with an equal volume of water with stirring or probe sonication.

Using these reprecipitation methods, in prototype experiments we produced nanoparticles in the range 50-300 nm, as determined by DLS, TEM, and SEM. We found that the particle size was a function of the dye concentration, the relative volume of water, and the aging time. The temperature and the sonication time likely have an effect as well, but experiments to determine the effect of varying those parameters had not yet been conducted as of the filing date of the present application.

In one experiment, we prepared MHIPF₆ dye nanoparticles with an average diameter of 118±37 nm by injecting 100 μL of a 1 mM solution of the dye in THF into 10 mL of water with vigorous stirring. The particles had high monodispersity in solution (PDI=0.087 from DLS).

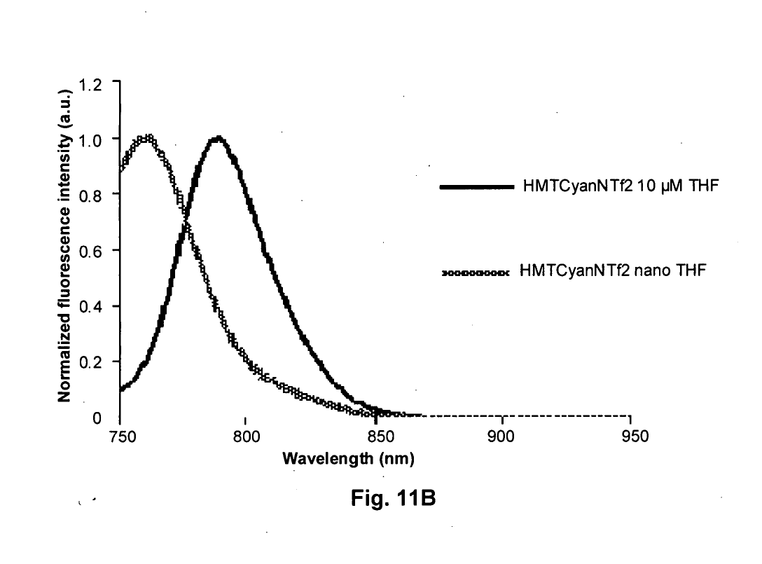

In another experiment we prepared HMTNTf₂ dye nanoparticles by reprecipitation. These particles appeared to be generally spherical when viewed by SEM. We measured their absorbance and fluorescence emission spectra (FIGS. 11(a) and (b), respectively), and compared them to spectra of the dye in solution. The absorbance and emission wavelengths for the particles were both blue-shifted as compared to those for the unmodified dye in solution. Optical microscopy confirmed that the particles indeed emitted light when excited at the long NIR wavelength.

### Example 21

### Fluorescent-Magnetic IL Microparticles and Nanoparticles

The novel microparticles and nanoparticles are “tunable,” meaning that their properties may be selected for particular purposes by appropriate choice of anion, cation, or both. As one example, the particles may be given fluorescent properties, or magnetic properties, or both. Previous functionalized magnetic particles have typically been based on a metal, metal hydride, or metal oxide core that is coated with functional groups. In a frozen IL nanoparticle or microparticle embodiment, however, magnetic particles may be made with single-component materials. The magnetic component need not be introduced as separate particles to be coated, but rather it may be introduced via a complex ion having a high magnetic moment. The resulting frozen IL particles can display a strong response to external magnetic fields.

We prepared fluorescent magnetic nanoparticles using the fluorescent dye Rhodamine B hydrochloride, and tetrachloroferrate (FeCl⁴⁻.6H₂O) at a 1:1 molar ratio in acetone. Total solute concentration was 0.1 g/mL. The mixture stirred at room temperature for 2 hours, and was then freeze-dried to remove solvent. The residual product (which was a solid at room temperature) was then used to prepare nanoparticles by aerosolization, following the method of Example 14. All IL bulk, solution, and particles retained their fluorescent properties as measured by a standard fluorometer. All IL bulk, solution, and particles were magnetic as tested by moving a granule/droplet with a 0.25 Tesla permanent magnet.

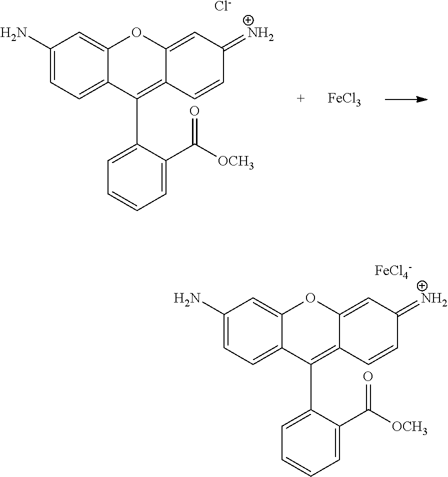

TABLE 3
Applications for ionic liquid nanoparticles.

| | Commonly-Used | |
|---|---|---|
| | Current | |
| Application | Techniques | Uses for Ionic Liquid Nanoparticles |
| Biomedical | Nanoparticles made of silica | The IL can be chosen for minimal quenching when doped with a |
| Imaging | or polymeric materials are | fluorophore. Alternatively, ILs can also be synthesized using a |
| | either tagged or doped with | fluorescent cation, a fluorescent anion, or both. Tuning is achieved by |
| | a dye for visualization and | surface activation of the particles to carry moieties with an affinity for |
| | detection of pathogens, | specific binding. Examples include ILs with cations such as RhodB, |
| | cancer cells, apoptosis, etc. | IR-797, CrystViol, BasicYellow, Methylviolet 2B, and MalachGreen |
| | | and anions such as lactate, amino acid esters, and nitrate. For affinity |
| | | binding to a target tissue to be visualized, antibodies may be chosen |
| | | that are specific for the target, e.g., cancer, apoptosis, atherosclerosis, |
| | | and other biological phenomena or disease protein markers. |
| Drug Delivery | Nanoparticles made of PLA, | The characteristics of the IL particles, such as biocompatibility, |
| | PLGA and other | biodegradability, and ability to entrap a drug, may be adjusted by |
| | biodegradable polymers are | selecting cations or anions such as amino acids, organic acids, or |
| | used for entrapping, | vitamins. Antibodies may be loaded on the particles for targeted- |
| | encapsulating and loading | delivery, for example by binding to the ε-NH₂ on lysine or to the —SH |
| | drugs on their surfaces for | group on cysteine. ILs can also be made using a drug molecule itself |
| | targeted or controlled | as the cation or anion. |
| | delivery or chemotherapy | |
| | agents, to bones, lungs, skin, | |
| | and other tissues. | |
| Analytical | Typically, porous | ILs can be chosen to have properties useful in separations, ranging |
| Separations | microparticles and non- | from simple hydrophilicity/hydrophobicity interactions to more |
| | porous nanoparticles are | complex properties such as enantioselectivity and other |
| | used as stationary phases in | characteristics. For example, Spiroimidazolium and other chiral |
| | liquid chromatography and | cations can be used in coordination with various anions for and chiral |
| | in capillary | IL for applications in capillary column packings for chiral |
| | electrochromatography. | separations. |
| | These stationary phases can | |
| | be coated or activated to | |
| | impart properties useful in a | |
| | separation, such as | |
| | hydrophobicity, protein | |
| | specificity, enantio- | |
| | selectivity, glycosyl affinity, | |
| | and other specific | |
| | selectivities. ILs have also | |
| | been used in affinity | |
| | chromatography as a liquid | |
| | component in the medium. | |
| Inks | Inks containing silver | A dye-IL or metal ion-containing IL may be used in an ink, e.g., ILs |
| | nanoparticles have been used | containing silver, such as silver lactate, and other metal-organic ILs. |
| | for agricultural or marine | |
| | products for assessing the | |
| | storage periods and | |
| | freshness. | |
| Sensory | Higher sensitivity and faster | IL nanoparticles with conductive, superconductive, or semiconductive |
| Devices and | response times are desirable | properties may be used in applications such as optical sensing, |
| Fuel Cells | properties in sensors and | electrochemical sensing, and fuel cells. Plastic ionic liquid crystal |
| | biosensors. Nanoparticles | phases may be formed with IL nano- and microparticles, which may |
| | provide a high surface area | be used as solid-state conductive materials or in fuel cells. |
| | to volume ratio, contributing | |
| | to an increase in sensitivity | |
| | and a reduction in the | |
| | response time. The small | |
| | size of nanoparticles helps in | |
| | toward the miniaturizing | |
| | sensory devices for | |
| | biomedical applications. | |
| Self-healing | Nanoparticles have been | The ability to reversibly melt and re-freeze (seal) IL-based materials, |
| | used in composite materials, | particularly at modest melting temperatures, offers a novel route to |
| | especially in optical fibers, | self-healing composites, and reversibly-conductive composites. |
| | to fill out cracks in “self- | |
| | healing” materials. Adding | |
| | nanoparticles to polymers | |
| | yields materials in which the | |
| | particles become localized at | |
| | nanoscale cracks and | |
| | effectively form “patches” | |
| | to repair the damaged | |
| | regions. | |
| Displays and | TiO₂, SiO₂ and Sb₂O₅ | IL crystals combine the unique solvent properties of ionic liquids with |
| Imaging | nanoparticles (7-20 nm) | the self-organization of liquid crystals. IL nanoparticles may be used |
| | have been used in liquid | in displays and imaging. Examples of ILs that should be useful for |
| | crystal displays employing | such applications include trioxadecyl-based, citronenyl-based, and |
| | polymer-dispersed liquid | trimethyldodecyl-based ILs. |
| | crystals (PDLCs) filled | |
| | with NPs. The nanoparticles | |
| | serve as building blocks for | |
| | the polymer matrix, | |
| | enhancing light scattering in | |
| | the polymer as well as | |
| | enhancing contrast. | |
| Inorganic, | Metal oxide nanoparticles | IL nanoparticles carrying metal oxides can be used in similar |
| Organic, and | have been used in | applications. Optionally, the IL can be chiral, such as a |
| Biological | heterogeneous catalysis, | Spiroimidazolium NTf₂ IL. |
| Catalysis | both in synthesizing more | |
| | complex compounds from | |
| | simpler ones, and in | |
| | breaking down more | |
| | complex compounds. For | |
| | example, they have been | |
| | used in the destruction of | |
| | nerve gas agents, in fuel cell | |
| | catalysis, and in the | |
| | breakdown of carbon | |
| | monoxide and nitric oxide | |
| | from cigarette smoke. | |
| | In some biocatalysis | |
| | applications it has been | |
| | shown that loading enzymes | |
| | or bacteria onto | |
| | nanoparticles can increase | |
| | the stability of the particles, | |
| | particularly at high | |
| | temperatures and in solution. | |
| Antimicrobial/ | Nanoparticles containing | ILs having antibiotic (including silver- or other metal-containing) |
| Antifouling | metal ions (especially silver | components, antiviral components, or both can be used. Examples |
| Nanoparticles | ions) often have | include tetracycline NTf₂ and other cationic antibiotics coupled with |
| | antimicrobial activity. Those | inorganic or organic anions. |
| | particles range from metal | |
| | colloids of a few nanometers | |
| | in diameter to surfaces of | |
| | mesoporous silica activated | |
| | with silver ions. | |
| Building | | (1) IL nanoparticles can be used as the organic component of organic- |
| Blocks for | | inorganic hybrid materials, followed by removal of one of the |
| Organic- | | components to produce novel templated materials. (2) Alternatively, |
| Inorganic | | the IL component can be retained as a conductive liquid phase held in |
| Hybrids | | a well-defined porous network. (3) As yet another alternative, the IL |
| | | nanoparticles can be maintained as solid-state encapsulants toward |
| | | environmentally responsive sensory or display devices. |
| Magnetic | Magnetic nanoparticles | Ionic liquids with FeCl4⁻ anion have shown to possess paramagnetic |
| | including Fe, FeₓO_{y}, and | characteristics. Example achieve were 1-butyl-methylimidazolium |
| | other metal and metal oxide | tetrafluoroferrite (bmim FeCl4). Particles derived out of those ionic |
| | particles such as magnetite | liquids have potential for catalysis, remediation, sensors and chemical |
| | (Fe₂O₃). | separations due to the ease of their recovery by simple extraction |
| | | using a magnet. |

### Miscellaneous

As used in the specification and claims, unless context clearly indicates otherwise, an “ionic liquid” is a salt having a melting point below about 200° C.; and in many cases is preferably below about 100° C., so that an aqueous solvent may be used in the synthesis. The term “ionic liquid” thus includes compositions that are, in fact, solids at temperatures below their respective melting points. The term does not imply that the salt is necessarily a liquid at any particular time; rather, it refers to the salt's melting point. Where an IL has a melting point above 100° C., higher boiling point solvents may be used such as glycerol, paraffin, mineral oil, and other solvents known in the art. Likewise, where a particular IL is water-soluble, then a nonaqueous solvent may be used for dispersal.

The melting point, according to the use for which the particles are intended, may be chosen to greater than or equal to about: 25° C., 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., or 195° C.

The melting point, according to the use for which the particles are intended, may be chosen to less than or equal to about: 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., or 200° C.

As used in the specification and claims, unless context clearly indicates otherwise, the term “fluid” should be understood to refer to fluid phases broadly, including gases, liquids, supercritical fluids, solutions, emulsions, colloids, aerosols, sols, and gels.

The “diameter” of a particle refers to the longest dimension across or through the particle, measured along a straight line. The use of the term “diameter” does not imply that a particle has any particular shape.

An “organic salt” is a salt comprising at least one organic anion, or at least one organic cation, or both an organic anion and an organic cation. Examples of organic ions that may be used include, for example,): tosylate, trifluoromethanesulfonate, tris (pentafluoroethyl)trifluorophosphate, bis(trifluoromethylsulfonyl)imide, lactate, tetraphenyl borate, 3,5-bis(trifluoromethyl)phenyltrifluoroborate, 4-(trifluoromethyl)phenyltrifluoroborate, tetrakis[3,5-bis(1,1,1,3,3,3-hexafluoro-2-methoxy-2-propyl)phenyl]borate, trifluorophenylborate, saccharin, acesulfame, fluorescein, eosin, and their respective derivatives.

Microparticles and nanoparticles in accordance with this invention have a diameter between about 1 nm and about 500 μm; preferably between about 10 nm and about 100 μm.

The diameter, according to the use for which the particles are intended, may be greater than or equal to about: 1 nm, 2 nm, 3 nm, 4 nm, 5 nm, 7 nm, 10 nm, 20 nm, 30 nm, 40 nm, 50 nm, 70 nm, 100 nm, 200 nm, 300 nm, 400 nm, 500 nm, 700 nm, 1 μm, 2 μm, 3 μm, 4 μm, 5 μm, 7 μm, 10 μm, 20 μm, 30 μm, 40 μm, 50 μm, 70 μm, 100 μm, 200 μm, 300 μm, 400 μm, or 500 μm.

The diameter, according to the use for which the particles are intended, may be less than or equal to about: 1 nm, 2 nm, 3 nm, 4 nm, 5 nm, 7 nm, 10 nm, 20 nm, 30 nm, 40 nm, 50 nm, 70 nm, 100 nm, 200 nm, 300 nm, 400 nm, 500 nm, 700 nm, 1 μm, 2 μm, 3 μm, 4 μm, 5 μm, 7 μm, 10 μm, 20 μm, 30 μm, 40 μm, 50 μm, 70 μm, 100 μm, 200 μm, 300 μm, 400 μm, or 500 μm.

In the particle range of 1-100 nm, IL nanoparticles will be useful in applications such as drug delivery, biomedical diagnostics, catalysis, displays and imaging, and as building blocks of organic and inorganic materials synthesis. Particles in the submicron range of 100 nm-1 μm can be used in inorganic catalysis, biomolecule carriers, analytical sensory devices, and affinity assays. Microparticles in the range of 1-500 μm can be used as packing materials in chromatography techniques, including both chiral and achiral separations. Microparticles may optionally be designed to be porous using techniques previously employed in sol-gel templating with triblock copolymers, dendrimers, and other pore templates. Porous microparticles will have very high surface areas, and thus will be effective in chromatography.

The IL melting point should be higher than any temperatures at which the ILs will be required to remain in the solid phase in particle form. In principle, there is no upper limit on what the melting point may be. As a practical matter, for many applications the melting point will be between about 25° C. and about 200° C. For convenience of handling and preparation, the melting point will often be between about 40° C. and about 100° C., a range that is appropriate for most of the applications discussed here.

The complete disclosures of all references cited in this specification are hereby incorporated by reference. Also incorporated by reference are the complete disclosures of U.S. provisional patent application 61/015,378, filed Dec. 20, 2007; U.S. provisional patent application 61/087,831, filed Aug. 11, 2008; A. Tesfai et al., “Controllable formation of ionic liquid micro- and nanoparticles via a melt-emulsion-quench approach,” Nano Letters, vol. 8, pp. 897-901 (2008); B. El-Zahab et al., “Frozen ionic liquids: A new breed of nanomaterials,” Abstract, 236th ACS National Meeting, Philadelphia, Pa., Aug. 17-21, 2008, IEC-184; I. Warner et al., “New directions in spectroscopy: Novel NIR dyes and new nanotechnology directions,” Abstract, 236th ACS National Meeting, Philadelphia, Pa., Aug. 17-21, 2008, ANYL-069; A. Tesfai et al., “Synthesis and Characterization of Novel Nano- and Micro-Particles,” Abstract, 35th Annual Conference of The National Organization of Black Chemists and Chemical Engineers, Philadelphia, Pa., Mar. 16-21, 2008, page 120; M. Lowry et al., “Surface Chemistry of Separations,” Abstract, 1st Zing Chemistry Conference: Trends in Surface Chemistry, Antigua and Barbuda, Jan. 7-10, 2008. In the event of an otherwise irreconcilable conflict, however, the present specification shall control.

What is claimed:

1. A particle wherein:
(a) said particle is solid-phase, and said particle has a diameter between about 1 nm and about 100 μm;
(b) said particle comprises an organic salt with a melting point above about 25° C.

2. A particle as in claim 1, wherein said particle has a diameter between about 10 nm and about 50 μm.

3. A particle as in claim 1, wherein said organic salt has a melting point below about 200° C.

4. A particle as in claim 1, wherein said organic salt has a melting point below about 100° C.

5. A particle as in claim 1, wherein said organic salt comprises an organic cation.

6. A particle as in claim 1, wherein said organic salt comprises an organic anion.

7. A particle as in claim 1, wherein said organic salt comprises an organic anion with antimicrobial or antiviral activity; or wherein said organic salt comprises an organic cation with antimicrobial or antiviral activity; or both.

8. A particle as in claim 1, wherein said organic salt comprises an organic anion with magnetic or fluorescent properties; or wherein said organic salt comprises an organic cation with magnetic or fluorescent properties; or both.

9. A composite particle that comprises a dendrimer core, and that also comprises an organic salt;
wherein both the dendrimer core and the organic salt have melting points above about 25° C.

10. A method comprising the steps of:
(a) preparing, at a first temperature, a first fluid comprising a solution of an organic salt, or comprising an emulsion of an organic salt, or comprising a melt of an organic salt;
wherein the salt has a melting point above about 25° C.; and
(b) rapidly dispersing the first fluid into a second fluid at a second temperature; wherein:
(c) the first fluid and the second fluid are different; or the first temperature and the second temperature or different; or both;
(d) the salt is insoluble in the second fluid at the second temperature; and
(e) the rate of dispersal of the first second into the second fluid at the second temperature is sufficiently rapid to cause the formation of a plurality of solid-phase particles of the salt.

11. The method of claim 10; wherein:
step (a) comprises forming an emulsion from a liquid and a salt, at a temperature above the melting point of the salt, wherein the salt is substantially insoluble in the liquid at the temperature of the emulsion; and
step (b) comprises cooling the emulsion to a temperature below the melting point of the salt, wherein the rate of said cooling is sufficiently rapid to cause the formation of solid particles.

12. The method of claim 11, wherein the emulsion additionally comprises a surfactant.

13. The method of claim 11, wherein the emulsion comprises micelles or reverse micelles containing the organic salt.

14. The method of claim 10, wherein the first fluid comprises an aerosol comprising a gas and a solution of the organic salt.

15. The method of claim 10; wherein the first fluid comprises a solution of the organic salt in a liquid in which the organic salt is soluble; wherein said dispersing step comprises forcing the first fluid through a plurality of nozzles into the second fluid; and wherein the second fluid comprises a liquid in which the organic salt is insoluble.

16. A process comprising electrospinning a mixture of a polymer, and an organic salt that has a melting point above about 25° C., under conditions suitable to produce nanofibers of the polymer and the organic salt.

17. A nanofiber prepared by the process of claim 16.
